(19) United States
(12) Patent Application Publication
(10) Pub. No.: US 2008/0095853 A1
(43) Pub. Date: Apr. 24, 2008

(54) Modified Release For Proton Pump Inhibitors

(76) Inventors: Niclas Clemmensen, Molndal (SE); Jan-Erik Lofroth, Molndal (SE); Katrin Walter, Molndal (SE); Peter Wang, Molndal (SE); Martin Wikberg, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 11/718,583

(22) PCT Filed: Nov. 2, 2005
(86) PCT No.: PCT/SE05/01642

§ 371 Date: May 3, 2007

Related U.S. Application Data
(60) Provisional application No. 60/625,621, filed on Nov. 4, 2004.

(51) Int. Cl. A61K 9/16; A61K 31/4439; A61P 1/00

(52) U.S. Cl. 424/490; 514/338

(57) ABSTRACT

An oral solid pharmaceutical dosage form comprising an acid sensitive proton pump inhibitor (PPI) as single active drug, releasing the PPI in two separate pulses, one immediate and one delayed. The PPI is formulated into a core material in the form of pellets, which are coated i.a. with a combination of a delayed release modifying layer and a lag time controlling layer. The pellets are further provided with an enteric coating layer. The application also relates to processes for preparing the dosage forms as well as their use in the treatment of gastrointestinal diseases.

### FIELD OF THE INVENTION

This invention relates to an oral solid pharmaceutical dosage form comprising an acid sensitive proton pump inhibitor (including combinations of proton pump inhibitors), as only active drug in enteric coated delayed release pellets, as well as a process for their manufacture and the use of such dosage forms in medical treatment of gastrointestinal disorders.

### BACKGROUND OF THE INVENTION AND PRIOR ART

Acid sensitive H⁺, K⁺-ATPase inhibitors also named as gastric proton pump inhibitors are for instance compounds known under the generic names omeprazole, lansoprazole, pantoprazole, rabeprazole, leminoprazole and esomeprazole. Some of these compounds are disclosed in EP-A1-0005129, EP-A1-124495, WO 94/27988, EP-A1-174726, EP-A1-166287 and GB 2163747.

These pharmaceutical substances are useful for inhibiting gastric acid secretion in mammals including man by controlling gastric acid secretion at the final step of the acid secretory pathway and thus reduce basal and stimulated gastric acid secretion irrespective of stimulus. In a more general sense, they may be used for prevention and treatment of gastric-acid related diseases in mammals and man, including e.g. reflux oesophagitis, gastritis, duodenitis, gastric ulcer, duodenal ulcer and Zollinger-Ellison syndrome. Furthermore, they may be used for treatment of other gastrointestinal disorders where gastric acid inhibitory effect is desirable e.g. in patients on NSAID therapy, in patients with Non Ulcer Dyspepsia, and in patients with symptomatic gastro-oesophageal reflux disease (GORD). They may also be used in patients in intensive care situations, in patients with acute upper gastrointestinal bleeding, pre- and post-operatively to prevent aspiration of gastric acid, to prevent post-operative nausea and vomiting (PONV), and treat stress ulceration. Further, they may be useful in the treatment of sleep disturbance, psoriasis as well as in the treatment of Helicobacter infections and diseases related to these.

Enteric coated formulations comprising a proton pump inhibitor (in the following also referred to as PPI), and formulations intended to deliver a PPI after a delayed period of time have earlier been reported. However, currently available formulations of PPIs still have some shortcomings and limitations. The efficacy of acid control during PPI treatment is greater during daytime and after meals than during the night, which may have therapeutic consequences. A recent US study showed that nocturnal heartburn affects nearly 80% of individuals with GERD, resulting in sleep disturbance in 75% of these patients. The consequence of this is an impaired daily function in many patients (Shaker et al, AM J Gastroentrol 2003; 98 (7): 1487-93). Furthermore, there are some type of patients for which a more intensive gastric acid inhibition than the conventional once daily treatment might be needed. It has been shown that nocturnal gastric acid suppression can be significantly improved by splitting a 40 mg esomeprazole dose into 20 mg bid. This treatment regimen provides both rapid and sustained acid suppression (Hammer et al, Alimentary Pharmacol Ther 2004; 19 (19): 1105-10).

The present invention claiming an oral dosage form comprising two PPI releasing portions has been developed with the aim to securing an effective acid control over the whole 24-hour period, thus removing the necessity for twice daily dosing. This will provide an aid of use and patient compliance. Such a modified release formulation would also result in a greater efficacy in acid secretion inhibition, especially at night, compared with the conventional formulations of PPIs.

EP 247983 (AB Hässle) describes dosage forms of omeprazole or an alkaline salt of omeprazole wherein the active ingredient together with an alkaline reacting compound is formulated into a core material having a subcoating layer disposed thereon and an enteric coating as the outer layer. The dosage forms are intended to release the active ingredient rapidly in the small intestines after passage of the acidic milieu of the stomach.

WO 9601623 and WO 9601624 (Astra AB) describe tableted dosage forms of omeprazole, esomeprazole and other proton pump inhibitors, wherein enteric coating layered pellets together with tablet excipients are compressed into a multiple unit tableted dosage form. It is essential in these tableted formulations that the enteric coating layer can withstand the compression forces during tabletting.

WO 9932093 A1 (Astra AB) discloses an enteric coated pharmaceutical dosage form comprising an H⁺, K⁺-ATPase inhibitor. The formulation comprises at least two portions of the H⁺, K⁺-ATPase inhibitor to be released in at least two consecutive pulses. At least one of the portion has a delayed release. Those pellets or tablets giving the delayed release pulse include a surrounding lag time controlling layer, which is a semipermeable membrane comprising a water resistant polymer, and which disrupts after a desired time. There is no disclosure of a combination of a delay release modifying layer and a lag time controlling layer, wherein the latter consists mainly of a high viscosity water soluble polymer.

U.S. Pat. No. 5,885,616 (Impax Pharmaceuticals Inc.) discloses a single bead drug delivery system which can provide a two-step release of an active agent to facilitate an immediate yet sustained drug delivery. It does not disclose a lag time controlling layer comprising a high viscosity water soluble polymers as the only or the essential polymer. Neither does it disclose or suggest this type of delivery system for PPI's.

WO 9819668 (Sharmatek) is directed to a multicompartnent delayed release drug delivery system for acid sensitive drugs like omeprazole. The delayed release is related to a delayed release enteric barrier providing gastro-resistant behaviour for delivering omeprazole in the proximal segment (pH 5-6) of the gastrointestinal tract. This enteric barrier comprises enteric coating polymers as material of this layer. There is no disclosure of a high viscosity water soluble polymer.

EP 1194131 B1 (Sanofi-Synthélabo) discloses a controlled release dosage form producing at least a timed pulse. The delayed release is achieved with a coating comprising one or more ammonio methacrylate copolymers (waterinsoluble polymers). The drug may be omeprazole. It does not disclose a lag time controlling layer comprising a high viscosity water soluble polymers as the only or the essential polymer. Neither does it disclose any delay release modifying layer according to the invention in the present application, nor any enteric coating layer.

WO 0158433 (Eurand) discloses a pharmaceutical dosage form such as a capsule, comprising a multitude of multicoated particulates as beads, pellets or granules. If the beads are not immediate release beads they have at least two coated membrane barriers. One of them is composed of an enteric polymer while the second membrane barrier is composed of a mixture of a water insoluble polymer and an enteric polymer. Further, they also have an optional intermediate membrane containing an acid. It does not disclose a lag time controlling layer comprising a high viscosity water soluble polymer as the only or the essential polymer. Neither does it disclose or suggest this delivery system for PPI's.

WO 0124777 (American Home Products) discloses a pharmaceutical formulation for once daily administration providing a phased release of a drug or particularly multiphase delivery of PPI's such as perprazole (nowadays known as esomeprazole). The core is surrounded by an outer semi-permeable membrane comprising a permeable water insoluble polymer and at least 50% by weight of glidant. The dosage form lacks an enteric coat. This patent application does not disclose a lag time controlling layer comprising a high viscosity water soluble polymer as the only or the essential polymer.

U.S. Pat. No. 6,749,867 B (Robinson, J. R. and McGinity, J. W.) presents a time-release dosage form for acid-sensitive drugs or more particularly omeprazole, including a drug-containing core surrounded by an inert time-release coating, being water soluble or water erodible, delaying release to generally 0.5-5.0 hours after administration. The formulation has no enteric coat.

WO 2000078293 A1 (AstraZeneca AB) presents a dosage form for omeprazole or an alkaline salt thereof, S-omeprazole or an alkaline salt thereof, as active ingredient in a core together with alkaline additive(s) and swelling agent(s). The core is coated with a semipermeable membrane, achieving a delayed release starting when the membrane disrupts. The polymers disclosed for use in the semipermeable membrane are water insoluble polymers. The formulations have no enteric coat.

EP 1086 694 A2 (Laboratorios Del Dr. Esteve, S. A.) presents a solid oral pharmaceutical formulation for acid sensitive benzimidazoles in the form of pellets. The pellets have at least a system for modified release that achieve slow release profiles by an intermediate layer comprising a combination of an inert, non-alkaline polymer insoluble in water (ethylcellulose) and an inert, non-alkaline polymer soluble in water (hydroxypropyl methyl cellulose). The slow release pellets can be mixed with fast release pellets and formulated into capsules or tablets.

WO 2002053097 A2 (Tap Pharmaceutical Products, Inc. USA) presents a non-enteric coated carrier for a proton pump inhibitor, including a bicarbonate or a carbonate salt of a Group IA metal.

None of these previously described formulations disclosed a dosage form having a combination of a delay release modifying layer and a lag time controlling layer, the latter comprising a high viscosity water soluble polymer, or discloses a dosage form having a dissolution pattern as described in this patent application.

There is still a need for a dosage form comprising an acid sensitive PPI in which formulation the PPI will be transported intact through the stomach and then after a further desired delay time the dose of the PPI will be rapidly released, together with a PPI portion that is rapidly released directly after the passage of the stomach without any further delay time.

One way to produce such formulations is to construct them as layered pellets. Pellets have advantageous properties in vivo compared to tablets, e.g. in respect of gastrointestinal transit properties, such as shorter residence time in the stomach and less variance of the same.

Manufacturing processes for layered pellets comprise most frequently some type of fluidized bed spraying processes. Problems experienced with this technique, especially when spraying a solution of a high viscosity hydrophilic polymer, is that the processing times are often too long for practical use.

### BRIEF DESCRIPTION OF THE INVENTION

The invention relates in one aspect to an oral solid pharmaceutical dosage form comprising as the single active drug an acid sensitive proton pump inhibitor (PPI), the dosage form comprises two PPI releasing portions, pellets releasing the PPI with a delayed release pulse and pellets releasing the PPI with an immediate release pulse, wherein the PPI is formulated into a core material in the form of pellets and the pellets giving the delayed release pulse have the following layers in the given order on the core material; a delay release modifying layer, a lag time controlling layer comprising as essential component a high viscosity water soluble polymer, an optional subcoating layer, and an outer enteric coating layer; in which dosage form said pellets are comprised together with a portion of pellets giving immediate release of the PPI, which have an optional subcoating layer and an outer enteric coating layer on the core material.

The immediate release is achieved as described earlier in the art, as immediate release enteric coated pellets/tablets or as quick dissolving layer on a tablet with the dissolution for this immediate portion only restricted by an enteric coat. The delayed release is achieved as described below and defined in the claims. Further information can be extracted from the Examples of the invention.

In a second aspect of the invention the oral solid pharmaceutical dosage form is comprising as the single active drug an acid sensitive proton pump inhibitor (PPI), the dosage form comprises one population of pellets with two PPI releasing portions, each pellets giving a delayed release pulse and an immediate release pulse, wherein the PPI is formulated into a core material in the form of pellets and the pellets having delayed release have the following layers in the given order on the core material; a delay release modifying layer, a lag time controlling layer comprising as essential component a high viscosity water soluble polymer, followed by a layer comprising a 2^{nd} PPI portion, an optional subcoating layer and an outer enteric coating layer.

The finalized dosage forms of the invention comprise as one element an immediate release is portion (releasing the PPI immediately after passing of the acidic milieu of the stomach) and as a second element a delayed release PPI portion, which after first passing the acidic milieu of the stomach and then is released after a further lag time (with negligable release) which is being in the range of 1-10 hours.

It has now surprisingly been found that the dosage forms of the invention have improved dissolution characteristics. These are that besides having a further delay (besides the one resulting from the enteric coating) the dissolution of the delayed pulse is more distinct than in prior art. This has been found to be an attribute of the combined delay release modifying layer and lag-time controlling layer.

This more distinct dissolution effect can be seen as an increased steepness for the dissolution curve for the delayed pulse once the dissolution commences.

The embodiments of the invention have a dissolution of PPI from the delayed pulse wherein the steepness is estimated as the average % per minute released of the drug, during the time elapsed between dissolution of 10% PPI until dissolution of 90% PPI (PPI in the delayed pulse). The PPI release is measured and the steepness can e.g. be graphically evaluated after measurement. The time period is usually less than approx. 130 minutes. For illustration, see FIG. 1. Measurement is done as described under the heading “Definitions” under “Detailed description of the invention”.

The acid sensitive proton pump inhibitors are formulated into pellet cores according to conventional methods, together with pharmaceutically acceptable excipients.

The pellet cores are coated with a delay release modifying layer before applying the lag time controlling layer.

This is accomplished by a further aspect of the invention, being a new inventive process for applying the lag-time controlling layer, in which process the core material comprising the acid sensitive proton pump inhibitor as single active ingredient (and coated with the delay release modifying layer) are coated with a high viscosity water soluble polymer (like e.g. hydroxypropyl methyl cellulose, also referred to as HPMC in the following, 4000 cps), in a dispersion. Using a dispersion of the high viscosity water soluble polymer makes the process advantageous in aspects like possibility of using higher concentration when spraying in a continuous mode, i.e. higher than compared with solutions, and possibility of using a higher spraying rate thereby giving a reduced processing time. This simplifies the process, makes it industrially more attractive and more economic than existing spraying techniques for these types of polymers.

Reported problems like clogging are also avoided, and thus there is a reduced need for addition of extra additives, e.g. anti-tacking agents.

Another advantage obtained with the new process is the improved release characteristic of the acid sensitive proton pump inhibitor from the products having the combination of a delay release modifying layer and a lag time controlling coat applied on the pellet cores before the outer enteric coating is applied.

A third aspect of the invention is to use an alkaline quality of the high viscosity water soluble polymer in the lag time controlling layer, such as e.g. hydroxypropyl methyl cellulose or of hydroxyethyl cellulose (the latter also referred to as HEC in the following). This gives i.a. stability advantages.

A double pulse dissolution is achieved either by mixing of the enteric coated delayed pulsed release pellets with enteric coated instant/immediate releasing pellets/tablets, the latter prepared according to the art (e.g. described in EP 247983, WO 9601623 and WO 9601624), and filling them into capsules or incorporating the mixture together with suitable tableting excipients into a tablet by compression, or by coating the lag-time coated cores with a further, second portion of the PPI in a fast releasing/dissolving layer, and before the final coating with an enteric coat, optionally preceded by a subcoating after the PPI comprising layer.

Doses foreseen to be used in the double pulsed embodiment of the invention is in the range of 2-500 mg divided into an immediate release portion and a delayed release portion of the acid sensitive proton pump inhibitor, suitably in combinations of e.g. equal doses e.g. 60 mg+60 mg, but doses divided into variable proportions are also contemplated, like e.g 40 mg+120 mg.

Doses foreseen, for the single delayed release pulse formulation embodiment, being comprised in the final preparation, are in the range of 1-400 mg.

The dosage forms are advantageously used to provide a method of treatment for Crohn's disease, gastric bleeding, ulcerous colitis, gastric ulcers, duodenal ulcers, gastroesoephagal reflux disease and the other diseases mentioned above.

### BRIEF DESCRIPTION OF THE DRAWINGS

FIG. 1 illustrates some of the definitions used in this application. See also the text in the part “Definitions” before the Examples.

FIG. 2 illustrates the release profile obtained from the embodiments obtained in Example 1.

FIG. 3 illustrates the release profile obtained from the embodiments obtained in Example 2.

FIG. 4 illustrates the release profile obtained from the embodiments obtained in Example 3

FIG. 5 illustrates the release profile obtained from the embodiments obtained in Example 4.

FIG. 6 illustrates the release profile obtained from the embodiments obtained in Example 5.

FIG. 7 illustrates the release profile obtained from the embodiments obtained in Example 6.

FIG. 8 illustrates the release profile obtained from the embodiments obtained in Example 7.

FIG. 9 illustrates the release profile obtained from the embodiments obtained in Example 8.

### DETAILED DESCRIPTION OF THE INVENTION

The dosage forms of the invention comprise an acid sensitive proton pump inhibitor (also referred to as PPI in the following) as only active drug.

In one special embodiment of the invention, the PPI in the immediate release pulse is another one than the PPI in the delayed release pulse. Still this dosage form comprises only PPI's as active drug.

These drugs, acid sensitive PPI's, are compounds of the general formula I, an alkaline salt thereof, one of the single enantiomers thereof or an alkaline salt of one of the enantiomers 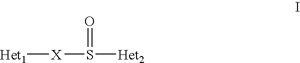
wherein
Het₁ is 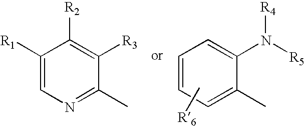
Het₂ is 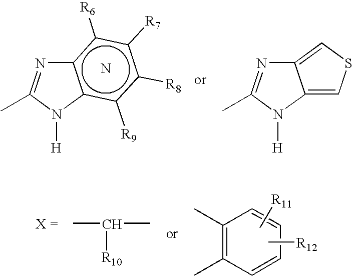
wherein
N in the benzimidazole moiety means that one of the ring carbon atoms substituted by R₆-R₉ optionally may be exchanged for a nitrogen atom without any substituents;
R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;
R₄ and R₅ are the same or different and selected from hydrogen, alkyl and arylalkyl;
R₆′ is hydrogen, halogen, trifluoromethyl, alkyl or alkoxy;
R₆-R₉ are the same or different and selected from hydrogen, alkyl, alkoxy, halogen, halo-alkoxy, alkylcarbonyl, alkoxycarbonyl, oxazolinyl, and trifluoroalkyl, or adjacent groups R₆-R₉ form ring structures which may be further substituted;
R₁₀ is hydrogen or forms an alkylene chain together with R₃ and
R₁₁ and R₁₂ are the same or different and selected from hydrogen, halogen or alkyl.

In the above definitions alkyl groups, alkoxy groups, and moieties thereof may be branched or straight C₁-C₉-chains or comprise cyclic alkyl groups, for example cycloalkylalkyl.

Examples of specifically interesting compounds according to formula I are

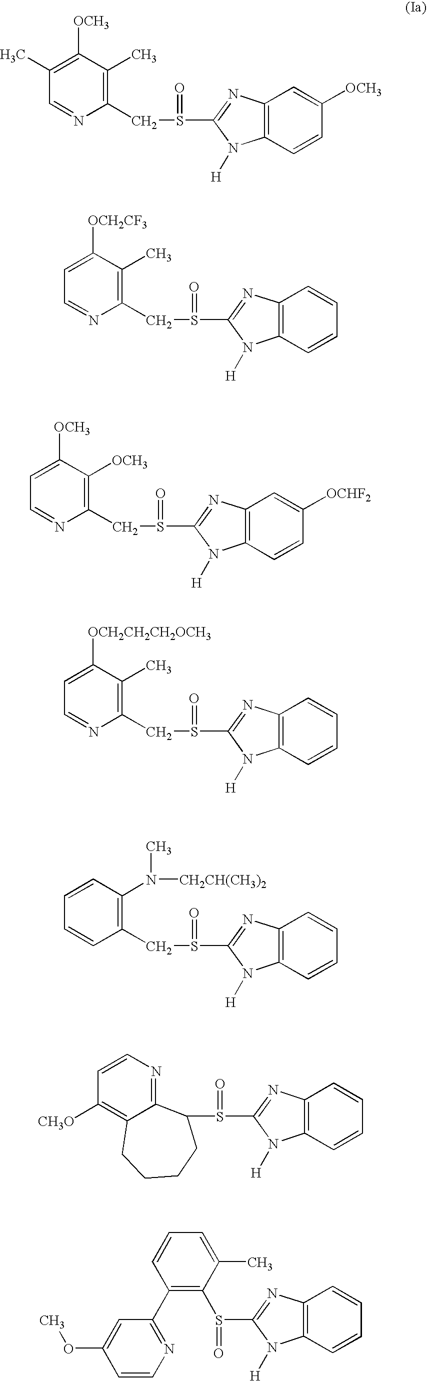

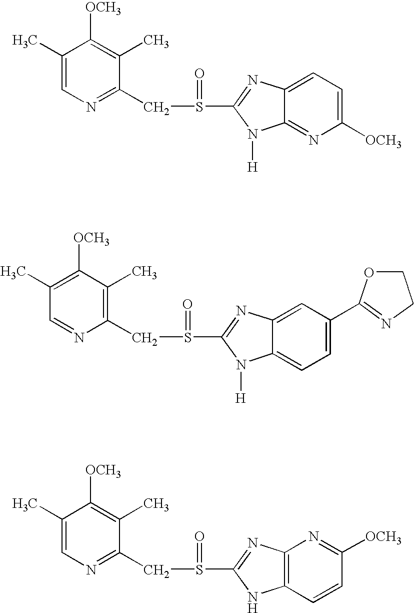

Preferred compounds for the oral pharmaceutical preparation according to the present invention are omeprazole, a magnesium salt of omeprazole or a magnesium salt of the (−)-enantiomer of omeprazole. The latter, the (−)-enantiomer of omeprazole, being named esomeprazole.

Especially preferred is an alkaline salt of esomeprazole, and most especially preferred is esomeprazole magnesium trihydrate.

In another embodiment of the invention tenatoprazole or one of its single enantiomers or a salt thereof, or a salt of tenatoprazole, is the active drug.

In a further special embodiment of the invention tenatoprazole or one of its single enantiomers or a salt thereof, or a salt of tenatoprazole, is the active drug in one pulse and another PPI is the active drug in the other pulse.

Doses

Doses foreseen to be used in the used double pulsed embodiment of the invention is in the range of 2-500 mg divided into one immediate release portion and one delayed release portion of the acid sensitive PPI, suitably in combinations of e.g. equal doses e.g. 60 mg+60 mg.

The invention also provides doses divided into variable proportions, like dividing the dose in proportions being 20%+80% of the total dose in one contemplated specific embodiment, in proportions being 30%+70% of the total dose in a 2nd contemplated specific embodiment and even further in proportions being 40%+60% in a third contemplated specific embodiment, without excluding any other possible dividing ratio between the immediate portion and the delayed release portion.

Doses foreseen, for the single delayed release pulse formulation embodiment, being comprised in the final preparation, are in the range of 1-400 mg. Preferably the dose is 2-200 mg, and most preferably the dose is 5-120 mg.

Core Material

The acid sensitive PPI comprising cores are formulated of the active drug optionally together with pharmaceutically acceptable excipients into a core material in the form of pellets according to conventional methods.

Among excipients in the cores may be mentioned, without restricting them to; diluents/fillers, pH regulating additives, disintegrants, osmotic agents, binders etc.

In a preferred embodiment the core material is exempt of acidic compounds. Acidic compounds according to this invention are compounds that give a pH of 5 or lower when dissolved or suspended in purified water at a concentration of 10% w/w (at room temperature, i.e. approx. 20 degrees Celsius), and measured with pH-meter equipped with a glass electrode or ISFET electrode.

In the case the core material is prepared by the layering technique, seeding materials can be chosen among but are not restricted to, water soluble particles as; Sugar seeds (USP), also known as non-pareils, salt crystals, etc, or water insoluble particles as; silicon dioxide, glass or plastic particles, microcrystalline cellulose (e.g. Celphere™) etc. Suitable types of insoluble plastic material are pharmaceutically acceptable plastics such as polypropylene or polyethylene. The preferred plastic material for seeding material is polypropylene. Also small particles of the active drug itself may be used as seeds.

Seeds have a size diameter in the range of 0.01-2 mm, preferably in the range of 0.2-0.8 mm. Another preferred alternative is 0.8-1.2 mm and a most preferred size diameter is in the range of 1.0-1.2 mm. The seeds are e.g. sprayed with a dispersion/solution/suspension of the active substance, together with a binder in a suitable coating apparatus, to obtain a core, with a seed having a deposited layer comprising the active drug.

A further preferred embodiment of the invention is that the diameter of the pellet cores is varied within a narrow distribution. Preferably the variation of the diameter in the population of pellets/beads is varied so that 90% by weight of the population is within +/− 10% of the average pellet diameter. This can be achieved by controlling the size of the starting materials, process parameters and/or by sieving. If the pellet cores are manufactured by the extrusion spheronization process, the amount of granulation liquid used can be one of the factors that influence the diameter obtained in the population of pellets. When the layering process is used, the size and size distribution of the starting seeds, e.g. non pareils or silicon dioxide seeds, is important in that aspect.

In one embodiment of the invention the pellet cores are sieved (after drying) to give a population of pellet cores in which 95% passes a sieve with 3.0 mm openings and in which 85% is retained on a sieve with 0.2 mm openings.

In a preferred embodiment of the invention the pellet cores are sieved (after drying) to give a population of pellet cores in which 95% passes a sieve with 2.0 mm openings and in which 85% is retained on a sieve with 0.5 mm openings.

In a most preferred embodiment of the invention the pellet cores are sieved (after drying) to give a population of pellet cores in which 95% passes a sieve with 1.6 mm openings and in which 85% is retained on a sieve with 1.2 mm openings.

Delay Release Modifying Layer

The delay release modifying layer that is applied onto the core material, and separates the lag time controlling layer from the PPI containing core is hydrophobized by incorporation of a hydrophobizing agent and talc in a water soluble polymer based layer.

Thus, the delay release modifying layer comprises a water soluble polymer(s), talc and a hydrophobizing agent which e.g. can be selected from the group consisting of Mg-stearate, glyceryl behenate and sodium stearyl fumarate.

Water soluble polymers in the delay release modifying layer are chosen to be solid polymers and have a viscosity below 180 mPas (cps).tested according to the European Pharmacopoeia. Also mixtures of such polymers are contemplated for use in the invention.

It is also important the delay release modifying layer does not include compounds having free acidic groups such as carboxylic acid groups or sulphonic acid groups in its composition, such as e.g. carbomers or enteric coating polymers. Thus, the release modifying layer is free from compounds having one or more free acidic group(s).

Examples of watersoluble polymers to be used include; Hydroxypropylcellulose, hydroxypropyl methyl cellulose, polyethylene glycol, polyvinyl alcohol, polyvinyl pyrrolidone, polyethylene-polypropylene glycol copolymers and the like.

The ratio between the water soluble polymer and talc is in the range of 1:1 to 1:8 (w/w), preferably in the range of 1:2 to 1:6 (w/w), and most preferably in the range of 1:3 to 1:4 (w/w).

The ratio between the water soluble polymer and the hydrophobizing compound is in the range of 3:1 to 5:1 (w/w), preferably 3.5:1 to 4.5:1 (w/w).

When the water soluble polymer in the delay release modifying layer is chosen to be hydroxypropyl cellulose (in the following also referred to as HPC), it is having a hydroxypropyl content in the range of 50-90% or more preferably in the range of 60-81%, and a viscosity below 180 mPas (cps) tested at 5% concentration. Such a polymer is, example given, Klucel LF from Aqualon.

The hydroxypropyl celluloses contemplated for use in this aspect of the invention, as a water soluble polymer in the delay release modifying layer, do not include Low-substituted hydroxypropyl cellulose, also referred to as L-HPC.

In a preferred embodiment of the invention the hydrophobizing agent is selected from the group consisting of Mg-stearate, glyceryl behenate and sodium steryl fumarate, or from mixtures thereof.

In one specific embodiment of the invention the watersoluble polymer is hydroxypropyl cellulose and the hydrophobizing compound is Mg-stearate.

In this embodiment of the invention the delay release modifying layer is only composed of the three excipients hydroxypropyl cellulose, talc and Mg-stearate, disregarding minor traces of solvents/water that may be remains from the coating process.

In this specific embodiment the ratio between HPC and talc is in the range of 1:1 to 1:8 (w/w), preferably in the range of 1:2 to 1:6 (w/w), and most preferably in the range of 1:3 to 1:4 (w/w).

Further, in the same specific embodiment the ratio between HPC and Mg-stearate is in the range of 3:1 to 5:1 (w/w), preferably 3.5:1 to 4.5:1 (w/w).

In an alternative specific embodiment of the invention the watersoluble polymer is hydroxypropyl cellulose and the hydrophobizing compound is Sodium stearyl fumarate.

Lag Time Controlling Layer

The lag time controlling layer comprises a high viscosity water soluble polymerlike e.g. hydroxypropylmethylcellulose 4000, as essential component. The term “a water soluble polymer” as used herein means a water soluble polymer, water soluble copolymer, or mixture of such polymers. With “high viscosity” in this invention is regarded an apparent viscosity of 100 mPas (cps) up to approx. 150 000 mPas (cps), tested according to as first alternative the European Pharmacopoeia and as second alternative the US Pharmacopoeia. In case of that tests are described in both pharmacopoeias, the method in the European one has prevalence.

In an alternative embodiment of this invention, the term high viscosity is regarding an apparent viscosity of 100 mPas (cps) up to approx. 5000 mPas (cps), tested according to as first alternative the European Pharmacopoeia and as second alternative the US Pharmacopoeia. In case of that tests are described in both pharmacopoeias, the method in the European one has prevalence.

The essential component, the high viscosity water soluble polymer, constitutes 51-100% w/w of the components forming the lag time controlling layer, i.e. after any solvents or dispersion/suspension media from the spraying solution/dispersion/suspension has been evaporated. Preferably the essential component constitutes 70-100% w/w of the lag time controlling layer, and more preferably the essential component constitutes 85-100% w/w of the lag time controlling layer.

In one alternative embodiment of the invention the lag time controlling layer comprises mixtures of high viscosity water soluble polymers.

In another alternative embodiment of the invention the lag time controlling layer only comprises high viscosity water soluble polymers of the same type but having different viscosities, disregarding trace amounts of solvents/water that may be remains from the coating process.

In a preferred alternative embodiment of the invention the lag time controlling layer comprises a moderately alkaline quality of one or more high viscosity water soluble polymer component, such as a moderately alkaline quality of HPMC or of HEC. With a moderately alkaline quality of a high viscosity water soluble polymer means a quality that gives a pH when measured according to Pharmacopoeia Europa between 7.0-9.0. This feature gives stability advantages to the dosage form.

In a further alternative embodiment of the invention the lag time controlling layer only comprises a single high viscosity water soluble polymer, i.e. the essential component constitutes 100% w/w of the lag time controlling layer, disregarding trace amounts of solvents/water that may be remains from the coating process. With a single polymer in this aspect, is considered a single polymer product, normally containing a limited range of polymer chain lengths distributed around an average value.

The total amount of lag time controlling layer applied onto the delay release modifying layered cores is chosen to effectuate the desired lag time (for the delayed release pulse) by testing the in-vitro dissolution.

The dosage forms of the invention are having one portion of the PPI with a lag time in the range of 1-10 hours preferably 1-8 hours or most preferably 1-6 hrs. In an alternative embodiment, the dosage forms of the invention are having one portion of the PPI with a lag time in the range of 2-10 hours, preferably 2-8 hours or most preferably 2-6 hours. In a further alternative embodiment, the dosage forms of the invention are having one portion of the PPI with a lag time in the range of 4-10 hours, preferably 4-8 hours or most preferably 4-6 hours.

The man skilled in the art understands the lag time can be controlled by the amount and viscosity of the water soluble polymer in the lag time controlling layer, such that an increase of both these variables results in an increase in lag time. He will also know that extensive lag times, i.e. longer than 10-12 hrs, not are interesting to achieve, as formulations are excreated from the human body with time, and that the benefit of therapy regimens longer than once daily is questionable. The illustrating examples of this invention gives some formulas for lag time controlling layer application, which are easily modified by the man skilled in the art if so desired.

A group of preferred water soluble polymers are cellulose derivatives, e g HPMC (hydroxypropyl methylcellulose), HEC (hydroxyethyl cellulose), HPC (hydroxypropyl cellulose) and other polysaccharides such as pectin and pectinates (e.g. calcium pectinate), locust bean gum, tragacanth gum, guar gum, gum arabic, tamarind gum, tara gum, carrageenan, water-soluble alginates, pullulan and synthetic polymers such as polyethyleneoxides, polyoxyethylene-polyoxypropylene copolymers (Pluronics®), or a mixture thereof. HEC polymers to be included in the invention also includes such viscosity grades when tested in 1% solution fulfills the above specified viscosity requirements for “high viscosity”. Non-limiting examples of such HEC grades are Natrosol 250 from Aqualon with the following type designations; HHX, HHR, H4R, HR, MHR, MR, KR, and GR.

Especially preferred high viscosity water-soluble polymers are polymers of the type HPMC, polyethyleneoxides, HEC, xanthan gums, guar gums, or mixtures thereof.

Most preferred high viscosity water soluble polymers are HPMC or HEC or mixtures thereof.

The lag time may be adjusted by the type of polymer or polymers mixed, and amount of polymer or polymers mixed, used in the delayed release controlling layer. Also the ratio between mixed polymer components in this layer may be used to adjust the lag time.

Optional Second Drug Comprising Layer for Pellet Cores

The previously described pellets having a lag time controlling layer, are as one alternative embodiment of the invention coated, e.g. sprayed, with a dispersion/solution/suspension of the PPI, together with a water soluble binder and optionally a surfactant. The coating is performed in a suitable coating apparatus, to obtain pellet cores having a 2^{nd} PPI comprising layer deposited on top of the lag time controlling layer, giving an immediate release pulse when the final preparation is administered.

Enteric Coating Layer(s) and Separating Layer(s).

Before applying an enteric coating layer onto the layered pellets, they may optionally be covered with one or more water soluble or in water rapidly disintegrating subcoating layers comprising pharmaceutical excipients optionally including alkaline compounds such as for instance pH-buffering compounds. This subcoating layer separates the composition of the layered pellets from the outer enteric coating layer.

The subcoating layer as well as the other type of layers, such as the lag time controlling layer, can be applied by coating or layering procedures in suitable equipments such as coating pan, coating granulator, centrifugal granulator or in a fluidized bed apparatus (including Wurster type) using water and/or organic solvents for the coating process. As an alternative the layer(s) can be applied by using powder coating technique.

Suitable materials for the optional separating layer are pharmaceutically acceptable compounds such as, for instance, sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc, pH-buffering substances and other additives may also be included into the subcoating layer.

When the optional subcoating layer is applied to the layered pellets or tablets it may constitute a variable thickness. The maximum thickness of the optional subcoating layer is normally only limited by processing conditions. The subcoating layer may serve as a diffusion barrier and may act as a pH-buffering zone. The optional subcoating layer may improve the chemical stability of the active substance and/or the physical properties of the dosage form.

Finally, the cores having a lag-time controlling layer and optionally a subcoating layer are covered by one or more enteric coating layers by using a suitable coating technique. The enteric coating layer material may be dispersed or dissolved in either water or in suitable organic solvents. As enteric coating layer polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethyl ethylcellulose, shellac or other suitable enteric coating layer polymer(s).

Additives such as dispersants, colorants, pigments, additional polymers e.g. poly(ethylacrylat, methylmethacrylat), anti-tacking and anti-foaming agents may also be included into the enteric coating layer. Other compounds may be added to increase film thickness and to decrease diffusion of acidic gastric juices into the acid susceptible material. The enteric coating layer(s) constitutes a thickness of approximately at least 10 μm, preferably more than 20 μm. The maximum thickness of the applied enteric coating layer(s) is normally only limited by processing conditions.

Any of the applied polymer containing layers, and specially the enteric coating layers may also contain pharmaceutically acceptable plasticizers to obtain desired mechanical properties. Such plasticizers are for instance, but not restricted to, triacetin, citric acid esters, phthalic acid esters, dibutyl sebacate, cetyl alcohol, polyethylene glycols, glycerol monoesters, polysorbates or other plasticizers. The amount of plasticizer is preferably optimized for each formula, in relation to the selected polymer(s), selected other additive(s) and the applied amount of said polymer(s).

In the alternative embodiment of the invention being enteric coated pellets that have no optional second PPI portion comprising layer (giving an immediate release pulse when administered) under the enteric coating layer, such pellets are mixed with immediate release pellets or tablets (of suitable size), the latter prepared according to the art, and formulated into capsules, sachets or multiple unit pellets system tablets. In such a way a final preparation giving both a delayed release pulse and an immediate release pulse of the PPI can be prepared.

Process

The final preparations of the present invention are made according to following principle process for the first alternative embodiment;

I) preparing a core material in the form of pellets comprising an acid sensitive proton pump inhibitor (PPI) as the only active drug;

II) coating the pellet cores obtained in step I) with a delay release modifying layer;

III) coating the delay release modifying layered pellet cores obtained from step II) with a lag time controlling layer comprising as essential component a high viscosity water soluble polymer;

IV) coating the lag-time controlling layered pellets obtained from step III) with an outer enteric coating, and an optional subcoating layer is applied before the enteric coating layer is applied;

V) incorporating the pellets product obtained in step IV) together with other pellets having an outer enteric coating and an optional subcoating layer, giving immediate release of the PPI, into a capsule, sachet, or multiple unit pellets system tablet.

The pellets giving immediate release are prepared according to the art, i.e. a core material comprising the PPI is layered with an enteric coating layer, and optional a subcoating layer is applied in between the core material and the enteric coating layer. These pellets giving an immediate release pulse is in one embodiment of the invention in the form of one or more tablet(s).

Optionally, the pellets product obtained in step IV) and pellets having an outer enteric coating and an optional subcoating layer, giving immediate release of the PPI, are mixed together before incorporation into a capsule, sachet, or tablet.

For the other alternative embodiment the final preparations are made according to the following process;

I) preparing a core material in the form of pellets comprising an acid sensitive proton pump inhibitor (PPI) as the only active drug;

II) coating the pellet cores obtained from step I) with a delay release modifying layer;

III) coating the delay release modifying layered pellet cores obtained from step II) with a lag time controlling layer comprising as essential component a high viscosity water soluble polymer;

IV) coating the lag-time controlling layered pellets obtained from step III) with a layer comprising a 2^{nd} PPI portion;

V) optionally coating the pellets obtained from step IV) with an optional subcoating layer; and

VI) coating the pellet product obtained from step V) with an outer enteric coating;

VII) formulating the enteric coated pellets obtained from step VI) into a capsule, sachet or multiple unit pellets system tablet.

For step II, for both alternative embodiments above, when coating the cores obtained in step I), it is especially beneficial to use a composition that gives a delay release modifying layer that only is composed of the ingredients hydroxypropyl cellulose, talc and Mg-stearate, except anysolvent/dispersant media/suspension media residues from the coating process.

For step III, for both alternative embodiments above, when coating the delay release modifying layered core from step II) it is especially beneficial to utilize a dispersion of the high viscosity water soluble polymer prepared by

a) dispersing the high viscosity water soluble polymer in a non-solvent; and

b) adding an aqueous liquid or water to form a hydrated form of the dispersed polymer particles;

It should be understood that such a dispersed system can not be obtained by first dissolving the polymer in a water-containing liquid and then precipitating the system.

Lag Times

The embodiments are designed for having a lag time for the delayed (second) pulse in the range of 1-10 hours, preferably 1-8 hours or most preferably 1-6 hours.

As an alternative the embodiments are designed for having a lag time in the range of 2-10 hours, preferably 2-8 hours or most preferably 2-6 hours.

As a further alternative the embodiments are designed for having a lag time in the range of 4-10 hours, preferably 4-8 hours or most preferably 4-6 hours.

In a preferred embodiment of the invention the embodiments are designed for having a lag time in the range of 1-10 hours, preferably 1-8 hours or most preferably 1-6 hours and a steepness of at least 0.6%/min₍₁₀₋₉₀₎ as characteristics for the delayed release portion of the drug or preferably the steepness is at least 1.0%/min₍₁₀₋₉₀₎.

As an alternative the preferred embodiments are designed for having a lag time in the range of 2-10 hours, preferably 2-8 hours or most preferably 2-6 hours and a steepness of at least 0.6%/min₍₁₀₋₉₀₎ as characteristics for the delayed release portion of the drug or preferably the steepness is at least 1.0%/min₍₁₀₋₉₀₎.

As a further alternative the preferred embodiments are designed for having a lag time in the range of 4-10 hours, preferably 4-8 hours or most preferably 4-6 hours and a steepness of at least 0.6%/min₍₁₀₋₉₀₎ as characteristics for the delayed release portion of the drug or preferably the steepness is at least 1.0%/min₍₁₀₋₉₀₎.

Final Dosage Forms

It is contemplated that the dosage forms of the invention before presentation to the patient is finalized to be in the form of capsules, sachets, or multiple unit pellet system tablets. The finalized dosage form may comprise alternative combinations of pellets, other type of pellets and tablets, giving the delayed release pulse respectively the immediate release pulse. The delayed release pulse is according to this invention originating from pellets. The following combinations are contemplated;

| | Comprising | | |
|---|---|---|---|
| | | Pellets second | |
| “Finalized” form | Pellets first type | type | Tablet(s) |
| Capsule | Delayed rel. | Immediate rel. | |
| Capsule | Delayed rel. | | Immediate rel. |
| Capsule | Delayed rel. + | | |
| | Immediate rel. | | |
| Tablet | Delayed rel. | Immediate rel. | |
| (multiple unit | | | |
| pellet system) | | | |
| Tablet | Delayed rel. + | | |
| (multiple unit | Immediate rel | | |
| pellet system) | | | |
| Sachet | Delayed rel. | Immediate rel. | |
| Sachet | Delayed rel. | | Immediate rel. |
| Sachet | Delayed rel. + | | |
| | Immediate rel. | | |

### DEFINITIONS

Lag time/delay time: means for this invention that the dissolution of PPI in vitro is delayed even after the enteric coated cores in form of pellets/tablets have been exposed for a first dissolution medium having pH 1.2 for 2 hours and then in a second dissolution medium having pH 6.8.

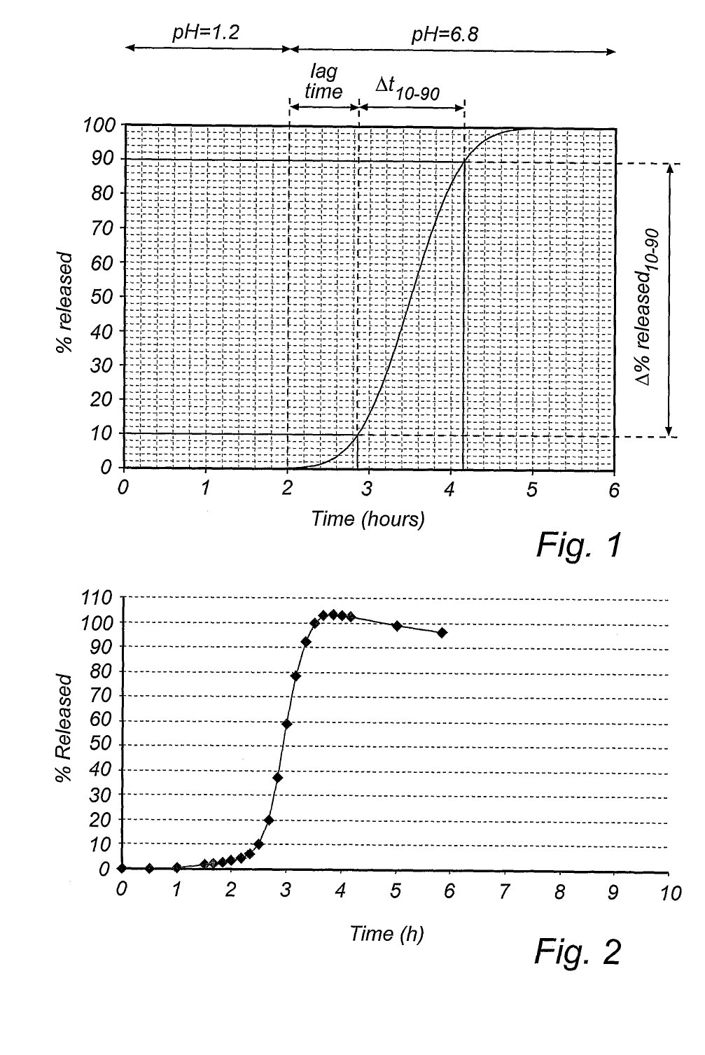

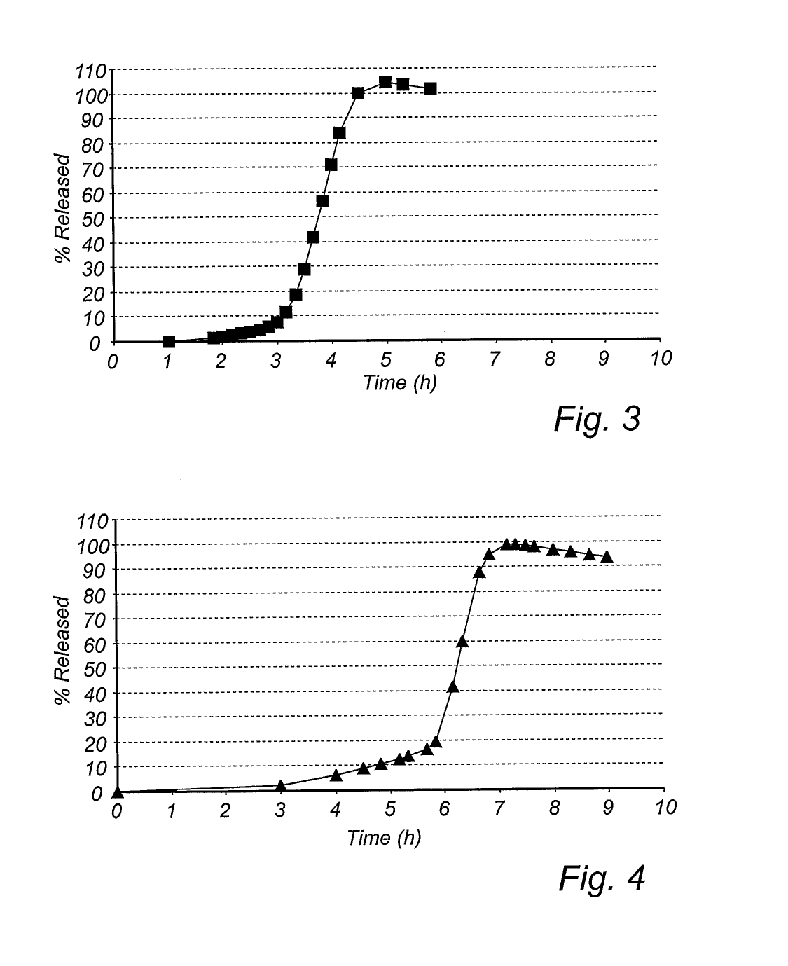

The lagtime is defined as the time in the (second) dissolution medium required until 10% of the drug (of the dose in the delayed pulse) is released. For illustration, see FIG. 1.

The dissolution is determined in vitro using a USP dissolution Apparatus No. 2 with paddle, as described in USP XXI, page 1244, at 37° C., operated at 100 rpm and using 300 ml 0.1 N hydrochloric acid as first dissolution medium and then 1000 ml phosphate buffer pH 6.8 as second dissolution medium. The amount released is measured spectrophotometrically as the absorption obtained in % of the absorption of a reference omeprazole sample at the same wavelength (302 nm). For other PPI's the wavelength may be adjusted to a more suitable one (which one can be determined by the man skilled in the art).

Steepness: the steepness is estimated as the average dissolution rate during the time elapsed between dissolution of 10% active drug until dissolution of 90% active drug (of the delayed dose). The drug release is measured and the steepness can e.g. be graphically evaluated after measurement.

The Steepness is defined as being the dissolved amount (80%) divided by the time in minutes required for dissolution of the 10-90% interval (of the delayed dose). This gives the Steepness as the average rate during this period as being expressed in % per minutes ₍₁₀₋₉₀₎. For illustration, see FIG. 1.

The steepness for the dosage forms of the invention is higher or equal to 0.6%/min₍₁₀₋₉₀₎. Preferably the dosage forms of the invention have a steepness of higher or equal to 1.0%/min₍₁₀₋₉₀₎.

The expression “negligible release” used in conjunction with the time period being the lag time, is less than 10% of the drug released.

The invention is illustrated by the following non-limiting examples.

### EXAMPLE 1

### Delayed Pulsed Release Pellets

All amounts given in compositions are charged amounts and not corrected for yields.

The schematic principle for the manufacture of the delayed pulsed release pellets was by coating seeds with layers in the following sequence;

Active drug (PPI) comprising layer→delay release modifying layer→lag time controlling layer→enteric coating layer.

| | |
|---|---|
| Excipients | Amount (g) |

Layering suspension for active drug (PPI) layer

| | |
|---|---|
| Esomeprazole-Mg trihydrate | 250 |
| Polysorbate 80 | 5.0 |
| Hydroxypropyl methyl cellulose 6 cps | 37.5 |
| Water purified | 1170 |

Seeds for active drug layering

| | |
|---|---|
| Sugar seeds (Non-pareil) 1.0-1.18 mm | 250 |

The layering suspension was prepared by the following procedure:

The hydroxypropyl methyl cellulose (in the following also referred to as HPMC) and the Polysorbate 80 were dissolved in the water whereafter the Esomeprazole-Mg trihydrate was suspended therein. The suspension was subjected to a wet micronizing step in an agitator mill (Dyno-Mill™).

The prepared layering suspension was spray-coated onto the sugar seeds in a fluidized bed equipment according to the Wurster principle, with a liquid nozzle having a 0.8 mm in diameter opening.

Inlet air temperature was 80° C., fluidizing air flow 40 m³/h, atomizer air pressure 2.5 bar, atomizer air flow 2.5 Nm³/h, spraying rate was 12-19 g/min resulting in an outlet air temperature of approx. 40° C.

500 g of the product from the first layering step was then coated with a delay release modifying layer solution/suspension prepared as described below:

Delay release modifying layer solution/suspension

| Excipients | Amount (g) |
|---|---|
| Talc powder | 112.5 |
| Hydroxypropyl cellulose (75-150 cps) | 30 |
| Mg-Stearate | 7.5 |
| Water purified | 1050 |

The hydroxypropyl cellulose was dissolved in the water. Thereafter the Talc and the Mg-Stearate was suspended therein.

The coating was performed in the same coating equipment as the preceding step. Inlet air temperature was 75° C., fluidizing air flow 40 m³/h, atomizer air pressure 2.8 bar, atomizer air flow 2.8 Nm³/h, spraying rate was 6-11 g/min resulting in an outlet air temperature of approx. 45° C.

180 g of the product from the delay release modifying layering step was then coated with a lag-time controlling layer by spraying a solution/suspension prepared as described below:

Solution/suspension for lag time controlling layer

| Excipients | Amount (g) |
|---|---|
| HPMC 4000 cps* | 80 |
| HPMC 6 cps | 11 |
| EtOH 99.5% | 1350 |
| Water purified | 172 |

*pH tested acc. to Pharm. Eur. to be 7.5

The high viscosity HPMC powder was suspended in the ethanol (non-solvent) while stirring. Under continued stirring a solution of the HPMC 6 cps and the water was gradually added, to result in low viscosity fluid comprising 91 g HPMC (polymer) per 1613 g total weight low viscosity fluid, i.e. concentration of 5.6% (w/w). The coating was performed in the same coating equipment as the preceding step. Inlet air temperature was 40° C., fluidizing air flow 40 m³/h, atomizer air pressure 2.5 bar, atomizer air flow 2.5 Nm³/h, spraying rate was 14-16 g/min resulting in an outlet air temperature of approx. 20° C.

150 g of the product from the lagtime controlling layer coating step was then coated with an enteric coating by spraying a suspension prepared as described below:

Enteric coating suspension

| Excipients | Amount (g) |
|---|---|
| Methacrylic acid copolymer type C, | 100 |
| 30% dispersion | |
| Talc | 6 |
| Triethyl citrate | 3 |
| Water purified | 126 |

The triethyl citrate was dissolved in the water while stirring. Under continued stirring the polymer dispersion was gradually added, and finally the talc was suspended in the dispersion.

The coating was performed in the same coating equipment as the preceeding step.

Inlet air temperature was 65° C., fluidizing air flow 40 m³/h, atomizer air pressure 2.8 bar, atomizer air flow 2.8 Nm³/h, spraying rate was 6-10 g/min resulting in an outlet air temperature of approx. 38° C.

A sample of the obtained product was tested for in vitro dissolution. The dissolution profile obtained is presented in FIG. 2.

The dissolution test was made in USP dissolution apparatus No. 2 with paddle, operated at 100 rpm. As dissolution media was used in the 2 hrs pre-exposure phase 300 ml 0.1 M HCl (37° C.) and then the medium was changed to 1000 ml phosphate buffer pH 6.8 (37° C.). The time in the pre-exposure medium is not reflected in the graph. Amount released esomeprazole magnesium measured by UV-spectroscopy at 302 nm. The declining end phase of the release curve (absorption value curve) may be attributed to some degradation of esomeprazole magnesium in the dissolution medium.

The lag time evaluated was between 2-2.5 hours, and the Steepness was approx. 1.0-1.1%/min₍₁₀₋₉₀₎.

### EXAMPLE 2

### Delayed Pulsed Release Pellets

All amounts given in compositions are charged amounts and not corrected for yields.

The schematic principle for the manufacture of the delayed pulsed release pellets was by coating seeds with layers in the following sequence; active drug (PPI) comprising layer→delay release modifying layer→lag time controlling layer→enteric coating layer.

Delay release modifying layered cores were obtained according to Ex. 1.

180 g of the product from the delay release modifying layering step was coated with a lag-time controlling layer by spraying a solution/suspension prepared as described below:

Solution/suspension for lag time controlling layer

| Excipients | Amount (g) |
|---|---|
| HPMC 4000 cps | 120 |
| HPMC 6 cps | 16.5 |
| EtOH 99.5% | 2025 |
| Water purified | 258 |

The high viscosity HPMC powder was suspended in the ethanol (non-solvent) while stirring. Under continued stirring a solution of the HPMC 6 cps and the water was gradually added, to result in low viscosity fluid comprising 136.5 g HPMC (polymer) in 2419.5 g total weight low viscosity fluid, i.e. concentration of 5.6% (w/w). The coating was performed in a fluidized bed equipment according to the Wurster principle, with a liquid nozzle having a 0.8 mm in diameter opening.

150 g of the product from the lagtime controlling layer coating step was then coated with an enteric coating by spraying a suspension prepared as described below:

Enteric coating suspension

| Excipients | Amount (g) |
|---|---|
| Methacrylic acid copolymer type C, | 100 |
| 30% dispersion | |
| Talc | 6 |
| Triethyl citrate | 3 |
| Water purified | 126 |

First the triethyl citrate was dissolved in the water while stirring. Under continued stirring the polymer dispersion was gradually added, and finally the talc was suspended in the dispersion.

The coating was performed in the same coating equipment as the preceding step.

A sample of the obtained product was tested for in vitro dissolution. The dissolution profile obtained is presented in FIG. 2.

The dissolution was tested as for Example 1

The lag time evaluated was approx. 2.5 hours. Steepness was approx. 1.0-1.1%/min₍₁₀₋₉₀₎.

### EXAMPLE 3

### Delayed Pulsed Release Pellets

All amounts given in compositions are charged amounts and not corrected for yields.

The schematic principle for the manufacture of the delayed pulsed release pellets was by coating seeds with layers in the following sequence; active drug (PPI) comprising layer→delay release modifying layer→lag time controlling layer→enteric coating layer.

Delay release modifying layered cores were obtained according to Ex. 1.

180 g of the product from the delay release modifying layering step was coated with a lag-time controlling layer by spraying a solution/suspension prepared as described below:

Solution/suspension for lag time controlling layer

| Excipients | Amount (g) |
|---|---|
| HPMC 4000 cps* | 240 |
| HPMC 6 cps | 33 |
| EtOH 99.5% | 4050 |
| Water purified | 516 |

*pH tested acc. to Pharm. Eur. to be 7.5

The high viscosity HPMC powder was suspended in the ethanol (non-solvent) while stirring. Under continued stirring a solution of the HPMC 6 cps and the water was gradually added, to result in low viscosity fluid comprising 273 g HPMC (polymer) in 4839 g total weight low viscosity fluid, i.e. a concentration of 5.6% (w/w).

The coating was performed in a fluidized bed equipment according to the Wurster principle, with a liquid nozzle having a 0.8 mm in diameter opening.

150 g of the product from the lagtime controlling layer coating step was then coated with an enteric coating by spraying a suspension prepared as described below:

Enteric coating suspension

| Excipients | Amount (g) |
|---|---|
| Methacrylic acid copolymer type C, | 100 |
| 30% dispersion | |
| Talc | 6 |
| Triethyl citrate | 3 |
| Water purified | 126 |

The triethyl citrate was dissolved in the water while stirring. Under continued stirring the polymer dispersion was gradually added, and finally the talc was suspended in the dispersion.

The coating was performed in the same coating equipment as the preceding step.

A sample of the obtained product was tested for in vitro dissolution. The dissolution profile obtained is presented in FIG. 4.

The dissolution test was made as in Ex. 1.

The lag time evaluated was approx. 4.5 hours. Steepness was approx. 0.6-0.7%/min₍₁₀₋₉₀₎.

### EXAMPLE 4

### Capsule Showing an Immediate Release Pulse and a Delayed Release Pulse of Esomeprazole Magnesium (40 mg+40 mg)

The schematic principle for the manufacture of the biphasic pulsed release capsules was by mixing pellets with immediate release and pellets with delayed release (i.e. pellets having the combined delay release modifying layer and lag time controlling layers according to the invention) and filling them into a capsule. I.e. the following sequence was followed;

preparing delayed release pellets (lag time pellets according to the invention)→mixing with immediate release pellets prepared accord. to prior art→filling into capsules.

| | Amount/ |
|---|---|
| Ingredients | capsule |
| Delayed Release pellets (from Exemple 1) | 238 mg |
| Immediate release pellets (from Nexium ® capsule) | 171 mg |
| Hard gelatin capsule (Size DBAA) | 1 piece |

175 capsules were made.

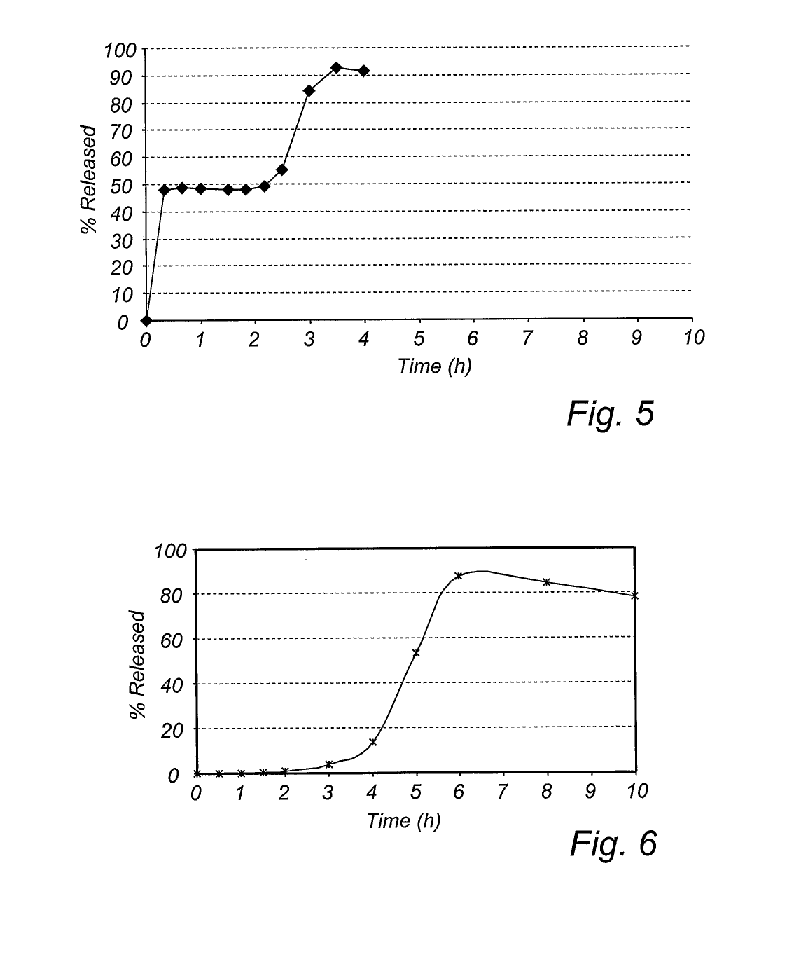

A sample of the obtained product was tested for in vitro dissolution. The dissolution profile obtained is presented in FIG. 5.

The dissolution was tested as in Example 1.

The lag time evaluated for the delayed release portion was approx. 2.5 hours, and steepness was approx. 1.3%/min₍₁₀₋₉₀₎.

Stability properties were investigated in a study, were the samples were kept in closed HDPE bottles with storage in 25° C. and 60% RH. The following results were obtained;

| | Amount of degradation |
|---|---|
| Time | products* |
| 0 | 0.2% |
| 1 year | 0.2% |

*Measured by HPLC as the sum of area for peaks of degradation products in relation to area of the omeprazole peak.

### EXAMPLE 5

### Delayed Pulsed Release Pellets

All amounts given in compositions are charged amounts and not corrected for yields.

The schematic principle for the manufacture of the delayed pulsed release pellets was by coating seeds with layers in the following sequence;

Active drug (PPI) comprising layer→delay release modifying layer→lag time controlling layer→subcoating layer→enteric coating layer.

| | |
|---|---|
| Excipients | Amount (g) |

Layering suspension for active drug layer

| | |
|---|---|
| Esomeprazole-Mg trihydrate | 300 |
| Polysorbate 80 | 6.0 |
| Hydroxypropyl methyl cellulose 6 cps | 45 |
| Water purified | 1404 |

Seeds for active drug layering

| | |
|---|---|
| Sugar seeds (Non-pareil) 1.0-1.18 mm | 300 |

The layering suspension was prepared by the following procedure:

The hydroxypropyl methyl cellulose and the Polysorbate 80 were dissolved in the water whereafter the esomeprazole-Mg trihydrate was suspended therein. The suspension was subjected to a wet micronizing step in an agitator mill (Dyno-Mill™).

The prepared layering suspension was spray-coated onto the sugar seeds in a fluidized bed equipment according to the Wurster principle, with a liquid nozzle having a 0.8 mm in diameter opening.

200 g of the product from the first layering step was then coated with a delay release modifying layer solution/suspension prepared as described below:

Delay release modifying layer solution/suspension

| Excipients | Amount (g) |
|---|---|
| Talc powder | 45 |
| Hydroxypropyl cellulose (75-150 cps) | 12 |
| Mg-Stearate | 3.0 |
| Water purified | 420 |

The hydroxypropyl cellulose was dissolved in the water. Thereafter the Talc and the Mg-Stearate were suspended therein.

The coating was performed in the same coating equipment as the preceding step.

The lag time controlling layer was applied in two operations onto the starting material from the preceeding step above, resulting in that 131 g starting material was coated with 240 g HPMC 4000 cps*(as the only polymer in this step), otherwise in analogy with previous examples (e.g. using the same solvent combination).

(* pH tested acc. to Pharm. Eur. to be 7.5).

The coating was performed in the same coating equipment as the preceding step.

150 g of the product from above was then coated with a subcoating suspension prepared as described below:

Subcoating suspension

| Excipients | Amount (g) |
|---|---|
| Talc powder | 25 |
| Hydroxypropyl cellulose (75-150 cps) | 6.7 |
| Mg-Stearate | 1.7 |
| Water purified | 234 |

The hydroxypropyl cellulose was dissolved in the water. Thereafter the Talc and the Mg-Stearate was suspended therein.

The coating was performed in the same coating equipment as the preceeding step.

150 g of the product from the sub coating step was then coated with an enteric coating by spraying a suspension prepared as described below:

Enteric coating suspension

| Excipients | Amount (g) |
|---|---|
| Methacrylic acid copolymer type C, | 100 |
| 30% dispersion | |
| Talc | 6 |
| Triethyl citrate | 3 |
| Water purified | 126 |

The triethyl citrate was dissolved in the water while stirring. Under continued stirring the polymer dispersion was gradually added, and finally the talc was suspended in the dispersion.

The coating was performed in the same coating equipment as the preceeding step.

A sample of the obtained product was tested for in vitro dissolution (as in Ex. 1). The dissolution profile obtained is presented in FIG. 6.

The lag time evaluated was approx. 4 hours. Steepness was approx. 0.7%/min₍₁₀₋₉₀₎.

Stability properties were investigated in an accelerated study, were the samples were kept in open storage in 40° C. and 75% RH. The following results were obtained;

| | Amount of degradation |
|---|---|
| Time | products* |
| 0 | 0.2-0.3% |
| 1 month | 0.2-0.3% |
| 2 months | 0.67% |

*Measured by HPLC as the sum of area for peaks of degradation products in relation to area of the omeprazole peak.

Samples were also kept in closed HDPE bottles in 25° C. and 60% RH. The following results were obtained;

| | Amount of degradation |
|---|---|
| Time | products* |
| 0 | 0.2-0.3% |
| 1 year | 0.2-0.3% |
| 2 years | 0.2-0.3% |

*Measured by HPLC as the sum of area for peaks of degradation products in relation to area of the omeprazole peak.

### EXAMPLE 6

### Delayed Pulsed Release Pellets

All amounts given in compositions are charged amounts and not corrected for yields.

The schematic principle for the manufacture of the delayed pulsed release pellets was by coating seeds with layers in the following sequence;

Active drug (PPI) comprising layer→delay release modifying layer→lag time controlling layer→enteric coating layer.

| | |
|---|---|
| Excipients | Amount (g) |

Layering suspension for active drug (PPI) layer

| | |
|---|---|
| Esomeprazole-Mg trihydrate | 250 |
| Polysorbate 80 | 5.0 |
| Hydroxypropyl methyl cellulose 6 cps | 37.5 |
| Water purified | 1170 |

Seeds for active drug layering

| | |
|---|---|
| Sugar seeds (Non-pareil) 1.0-1.18 mm | 250 |

The layering suspension was prepared by the following procedure:

The hydroxypropyl methyl cellulose and the Polysorbate 80 were dissolved in the water whereafter the Esomeprazole-Mg trihydrate was suspended therein. The suspension was subjected to a wet micronizing step in an agitator mill (Dyno-Mill™).

The prepared layering suspension was spray-coated onto the sugar seeds in a fluidized bed equipment according to the Wurster principle, with a liquid nozzle having a 0.8 mm in diameter opening.

Inlet air temperature was 80° C., fluidizing air flow 40 m³/h, atomizer air pressure 2.5 bar, atomizer air flow 2.5 Nm³/h, spraying rate was 12-19 g/min resulting in an outlet air temperature of approx. 40° C.

150 g of the product from the first layering step was then coated with a delay release modifying layer solution prepared as described below:

Delay release modifying layer solution/suspension

| Excipients | Amount (g) |
|---|---|
| Talc powder | 20.0 |
| Hydroxypropyl cellulose (75-150 cps) | 9.0 |
| Sodium Stearylfumarate (Pruv ®) | 2.3 |
| Water purified | 250 |

The hydroxypropyl cellulose was dissolved in the water. Thereafter the Talc and the Sodium-Stearylfumarate were suspended therein. The coating was performed in the same coating equipment as the preceding step.

Inlet air temperature was 75° C., fluidizing air flow 40 m³/h, atomizer air pressure 2.8 bar, atomizer air flow 2.8 Nm³/h, spraying rate was 6-11 g/min resulting in an outlet air temperature of approx. 45° C.

173 g of the product from the delay release modifying layering step was then coated with a lag-time controlling layer by spraying a solution/suspension prepared as described below:

Solution/suspension for lag time controlling layer

| Excipients | Amount (g) |
|---|---|
| HPMC 4000 cps | 115.5 |
| HPMC 6 cps | 15.9 |
| EtOH 99.5% | 1950 |
| Water purified | 248 |

The high viscosity HPMC powder was suspended in the ethanol (non-solvent) while stirring. Under continued stirring a solution of the HPMC 6 cps and the water was gradually added.

The coating was performed in the same coating equipment as the preceding step. Inlet air temperature was 40° C., fluidizing air flow 40 m³/h, atomizer air pressure 2.5 bar, atomizer air flow 2.5 Nm³/h, spraying rate was 14-16 g/min resulting in an outlet air temperature of approx. 20° C.

150 g of the product from the lagtime controlling layer coating step was then coated with an enteric coating by spraying a suspension prepared as described below:

Enteric coating suspension

| Excipients | Amount (g) |
|---|---|
| Methacrylic acid copolymer type C, 30% | 100 |
| dispersion | |
| Talc | 6 |
| Triethyl citrate | 3 |
| Water purified | 126 |

The triethyl citrate was dissolved in the water while stirring. Under continued stirring the polymer dispersion was gradually added, and finally the talc was suspended in the dispersion.

The coating was performed in the same coating equipment as the preceeding step.

Inlet air temperature was 65° C., fluidizing air flow 40 m³/h, atomizer air pressure 2.8 bar, atomizer air flow 2.8 Nm³/h, spraying rate was 6-10 g/min resulting in an outlet air temperature of approx. 38° C.

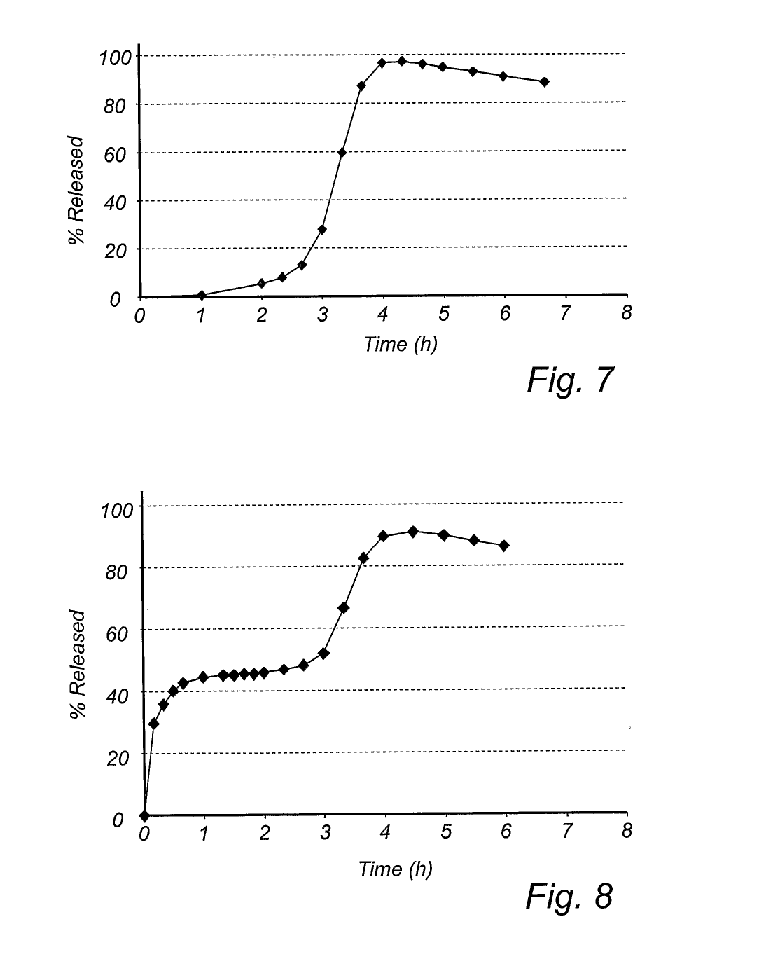

A sample of the obtained product was tested for in vitro dissolution. The dissolution profile obtained is presented in FIG. 7.

The dissolution test was made as described in Ex. 1.

The lag time evaluated was approx. 2.5 hours, and the Steepness was approx. 1.0-1.1%/min₍₁₀₋₉₀₎.

### EXAMPLE 7

### Delayed Pulsed Release Pellets with Two Pulses Separated in Time

All amounts given in compositions are charged amounts and not corrected for yields.

The schematic principle for the manufacture of the delayed pulsed release pellets was by coating seeds with layers in the following sequence;

Active drug (PPI) comprising (first) layer→delay release modifying layer→lag time controlling layer→Active drug (PPI) comprising (second) layer→subcoating layer→enteric coating layer.

| | |
|---|---|
| Excipients | Amount (g) |

Layering suspension for first active drug (PPI) layer

| | |
|---|---|
| Esomeprazole-Mg trihydrate | 250 |
| Polysorbate 80 | 5.0 |
| Hydroxypropyl methyl cellulose 6 cps | 37.5 |
| Water purified | 1170 |

Seeds for active drug layering

| | |
|---|---|
| Sugar seeds (Non-pareil) 1.0-1.18 mm | 250 |

The layering suspension was prepared by the following procedure:

The hydroxypropyl methyl cellulose (in the following also referred to as HPMC) and the Polysorbate 80 were dissolved in the water whereafter the Esomeprazole-Mg trihydrate was suspended therein. The suspension was subjected to a wet micronizing step in an agitator mill (Dyno-Mill™).

The prepared layering suspension was spray-coated onto the sugar seeds in a fluidized bed equipment according to the Wurster principle, with a liquid nozzle having a 0.8 mm in diameter opening.

Inlet air temperature was 80° C., fluidizing air flow 40 m³/h, atomizer air pressure 2.5 bar, atomizer air flow 2.5 Nm³/h, spraying rate was 12-19 g/min resulting in an outlet air temperature of approx. 40° C.

500 g of the product from the first layering step was then coated with a delay release modifying layer solution/suspension prepared as described below:

Delay release modifying layer solution/suspension

| Excipients | Amount (g) |
|---|---|
| Talc powder | 112.5 |
| Hydroxypropyl cellulose (75-150 cps) | 30 |
| Mg-Stearate | 7.5 |
| Water purified | 1050 |

The hydroxypropyl cellulose was dissolved in the water. Thereafter the Talc and the Mg-Stearate were suspended therein.

The coating was performed in the same coating equipment as the preceding step. Inlet air temperature was 75° C., fluidizing air flow 40 m³/h, atomizer air pressure 2.8 bar, atomizer air flow 2.8 Nm³/h, spraying rate was 6-11 g/min resulting in an outlet air temperature of approx. 45° C.

180 g of the product from the delay release modifying layering step was coated with a lag time controlling layer by spraying a solution/suspension prepared as described below:

Solution/suspension for lag time controlling layer

| Excipients | Amount (g) |
|---|---|
| HPMC 4000 cps | 120 |
| HPMC 6 cps | 16.5 |
| EtOH 99.5% | 2025 |
| Water purified | 258 |

The high viscosity HPMC powder was suspended in the ethanol (non-solvent) while stirring. Under continued stirring a solution of the HPMC 6 cps and the water was gradually added. The coating was performed in a fluidized bed equipment according to the Wurster principle, with a liquid nozzle having a 0.8 mm in diameter opening.

200 g of the product obtained from the step of applying the lag time controlling layer according to above, was coated with a second active drug (PPI) layer, by spraying a solution/suspension prepared as described below:

Layering suspension for second active drug (PPI) layer

| Excipients | Amount (g) |
|---|---|
| Omeprazole powder micronized | 40 |
| Polysorbate 80 | 0.8 |
| Hydroxypropyl methyl cellulose 6 cps | 6 |
| Water purified | 187 |

The layering suspension was prepared by the following procedure:

The hydroxypropyl methyl cellulose and the Polysorbate 80 were dissolved in the water whereafter the Omeprazole powder was suspended therein.

The prepared layering suspension was spray-coated onto the earlier obtained pellets according to above, in the same fluidized bed equipment.

Inlet air temperature was 80° C., fluidizing air flow 40 m³/h, atomizer air pressure 2.5 bar, atomizer air flow 2.5 Nm³/h, spraying rate was 10-13 g/min resulting in an outlet air temperature of approx. 40° C.

200 g of the product obtained from the step of applying the second active drug layer according to above, was coated with a subcoat, by spraying a solution/suspension prepared as described below:

Subcoating layer suspension

| Excipients | Amount (g) |
|---|---|
| Talc powder | 37.5 |
| Hydroxypropyl cellulose (75-150 cps) | 10 |
| Magnesium Stearate | 2.5 |
| Water purified | 350 |

The hydroxypropyl cellulose was dissolved in the water. Thereafter the Talc and the Magnesium Stearate were suspended therein.

The coating was performed in the same coating equipment as the preceding step.

Inlet air temperature was 75° C., fluidizing air flow 40 m³/h, atomizer air pressure 2.8 bar, atomizer air flow 2.8 Nm³/h, spraying rate was 6-11 g/min resulting in an outlet air temperature of approx. 45° C.

150 g of the product obtained from the step above, was coated with an enteric coating layer, by spraying a solution/suspension prepared as described below:

Enteric coating suspension

| Excipients | Amount (g) |
|---|---|
| Methacrylic acid copolymer type C, 30% | 100 |
| dispersion | |
| Talc | 6 |
| Triethyl citrate | 3 |
| Water purified | 126 |

First the triethyl citrate was dissolved in the water while stirring. Under continued stirring the polymer dispersion was gradually added, and finally the talc was suspended in the dispersion.

The coating was performed in the same coating equipment as the preceding step.

A sample of the obtained product was tested for in vitro dissolution. The dissolution profile obtained is presented in FIG. 8.

The dissolution was tested as for Example 1

The lag time for the second pulse evaluated was approx. 3 hours. Steepness was approx. 1.4%/min₍₁₀₋₉₀₎.

### EXAMPLE 8

### Delayed Pulsed Release Pellets

All amounts given in compositions are charged amounts and not corrected for yields.

The schematic principle for the manufacture of the delayed pulsed release pellets was by coating seeds with layers in the following sequence;

Active drug (PPI) comprising layer→delay release modifying layer→lag time controlling layer→enteric coating layer.

| | |
|---|---|
| Excipients | Amount (g) |

Layering suspension for the active drug (PPI) layer

| | |
|---|---|
| Esomeprazole-Mg trihydrate | 250 |
| Polysorbate 80 | 5.0 |
| Hydroxypropyl methyl cellulose 6 cps | 37.5 |
| Water purified | 1170 |

Seeds for active drug layering

| | |
|---|---|
| Sugar seeds (Non-pareil) 1.0-1.18 mm | 250 |

The layering suspension was prepared by the following procedure:

The hydroxypropyl methyl cellulose and the Polysorbate 80 were dissolved in the water whereafter the Esomeprazole-Mg trihydrate was suspended therein. The suspension was subjected to a wet micronizing step in an agitator mill (Dyno-Mill™).

The prepared layering suspension was spray-coated onto the sugar seeds in a fluidized bed equipment according to the Wurster principle, with a liquid nozzle having a 0.8 mm in diameter opening.

Inlet air temperature was 80° C., fluidizing air flow 40 m³/h, atomizer air pressure 2.5 bar, atomizer air flow 2.5 Nm³/h, spraying rate was 12-19 g/min resulting in an outlet air temperature of approx. 40° C.

500 g of the product from the first layering step was then coated with a delay release modifying layer solution/suspension prepared as described below:

Delay release modifying layer solution/suspension

| Excipients | Amount (g) |
|---|---|
| Talc powder | 112.5 |
| Hydroxypropyl cellulose (75-150 cps) | 30 |
| Mg-Stearate | 7.5 |
| Water purified | 1050 |

The hydroxypropyl cellulose was dissolved in the water. Thereafter the Talc and the Mg-Stearate was suspended therein.

The coating was performed in the same coating equipment as the preceding step.

Inlet air temperature was 75° C., fluidizing air flow 40 m³/h, atomizer air pressure 2.8 bar, atomizer air flow 2.8 Nm³/h, spraying rate was 6-11 g/min resulting in an outlet air temperature of approx. 45° C.

200 g of the product from the delay release modifying layering step was coated with a lag-time controlling layer by spraying a solution/suspension prepared as described below:

Solution/suspension for lag time controlling layer

| Excipients | Amount (g) |
|---|---|
| Hydroxy ethyl cellulose (Natrosol 250 HHX ®), | 90 |
| sieved <100 μm | |
| HPMC 6 cps | 13.5 |
| EtOH 99.5% | 900 |
| Water purified | 212 |

The Hydroxy ethyl cellulose powder was suspended in the ethanol (non-solvent) while stirring. Under continued stirring a solution of the HPMC 6 cps and the water was gradually added.

The coating was performed in a fluidized bed equipment according to the Wurster principle, with a liquid nozzle having a 0.8 mm in diameter opening.

150 g of the product obtained from the step above, was coated with an enteric coating layer, by spraying a solution/suspension prepared as described below:

Enteric coating suspension

| Excipients | Amount (g) |
|---|---|
| Methacrylic acid copolymer type C, | 100 |
| 30% dispersion | |
| Talc | 6 |
| Triethyl citrate | 3 |
| Water purified | 126 |

First the triethyl citrate was dissolved in the water while stirring. Under continued stirring the polymer dispersion was gradually added, and finally the talc was suspended in the dispersion.

The coating was performed in the same coating equipment as the preceding step.

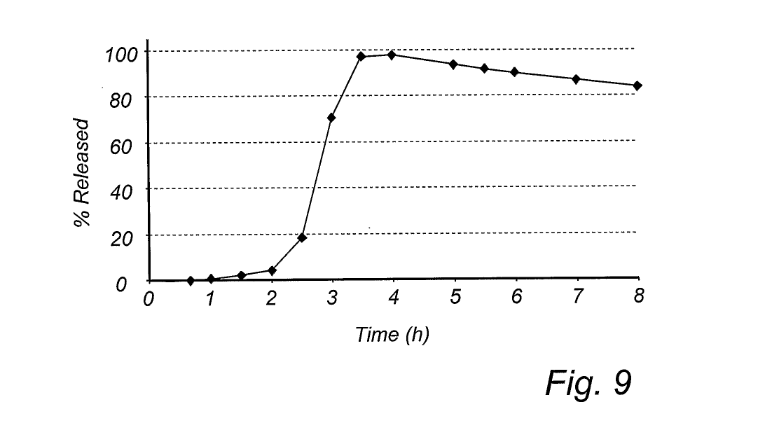

A sample of the obtained product was tested for in vitro dissolution. The dissolution profile obtained is presented in FIG. 9.

The dissolution was tested as for Example 1

The lag time for the second pulse evaluated was approx. 2 hours. Steepness was approx. 1.2%/min₍₁₀₋₉₀₎.

### EXAMPLE 9

### Delayed Pulsed Release Lansoprazole Pellets

All amounts given in compositions are charged amounts and not corrected for yields.

The schematic principle for the manufacture of the delayed pulsed release pellets is by coating seeds with layers in the following sequence;

Active drug (PPI) comprising layer→delay release modifying layer→lag time controlling layer→enteric coating layer.

| | |
|---|---|
| Excipients | Amount (g) |

Layering suspension for active drug (PPI) layer

| | |
|---|---|
| Lansoprazole | 250 |
| Polysorbate 80 | 5.0 |
| Hydroxypropyl methyl cellulose 6 cps | 37.5 |
| Water purified | 1170 |

Seeds for active drug layering

| | |
|---|---|
| Sugar seeds (Non-pareil) 1.0-1.18 mm | 250 |

The layering suspension is prepared by the following procedure:

The hydroxypropyl methyl cellulose and the Polysorbate 80 are dissolved in the water whereafter the Lansoprazole is suspended therein. The suspension is subjected to a wet micronizing step in an agitator mill (Dyno-Mill™).

The prepared layering suspension is spray-coated onto the sugar seeds in a fluidized bed equipment according to the Wurster principle, with a liquid nozzle having a 0.8 mm in diameter opening.

Inlet air temperature is set to 80° C., fluidizing air flow 40 m³/h, atomizer air pressure 2.5 bar, atomizer air flow 2.5 Nm³/h, spraying rate to 12-19 g/min.

500 g of the product from the first layering step is then coated with a delay release modifying layer solution/suspension prepared as described below:

Delay release modifying layer solution/suspension

| Excipients | Amount (g) |
|---|---|
| Talc powder | 112.5 |
| Hydroxypropyl cellulose (75-150 cps) | 30 |
| Mg-Stearate | 7.5 |
| Water purified | 1050 |

The hydroxypropyl cellulose is dissolved in the water. Thereafter the Talc and the Mg-Stearate are suspended therein.

The coating is performed in the same coating equipment as the preceeding step.

Inlet air temperature is set to 75° C., fluidizing air flow 40 m³/h, atomizer air pressure 2.8 bar, atomizer air flow 2.8 Nm³/h, spraying rate to 6-11 g/min.

180 g of the product from the delay release modifying layering step is then coated with a lag-time controlling layer by spraying a solution/suspension prepared as described below:

Solution/suspension for lag time controlling layer

| Excipients | Amount (g) |
|---|---|
| HPMC 4000 cps | 80 |
| HPMC 6 cps | 11 |
| EtOH 99.5% | 1350 |
| Water purified | 172 |

The high viscosity HPMC powder is suspended in the ethanol (non-solvent) while stirring. Under continued stirring a solution of the HPMC 6 cps and the water is gradually added.

The coating is performed in the same coating equipment as the preceding step.

Inlet air temperature is set to 40° C., fluidizing air flow 40 m³/h, atomizer air pressure 2.5 bar, atomizer air flow 2.5 Nm³/h, spraying rate to 14-16 g/min.

150 g of the product from the lagtime controlling layer coating step is then coated with an enteric coating by spraying a suspension prepared as described below:

Enteric coating suspension

| Excipients | Amount (g) |
|---|---|
| Methacrylic acid copolymer type C, | 100 |
| 30% dispersion | |
| Talc | 6 |
| Triethyl citrate | 3 |
| Water purified | 126 |

The triethyl citrate is dissolved in the water while stirring. Under continued stirring the polymer dispersion is gradually added, and finally the talc is suspended in the dispersion. The coating is performed in the same coating equipment as the preceeding step. Inlet air temperature is set to 65° C., fluidizing air flow 40 m³/h, atomizer air pressure 2.8 bar, atomizer air flow 2.8 Nm³/h, spraying rate to 6-10 g/min.

1. An oral solid pharmaceutical dosage form comprising:
(i) an acid sensitive proton pump inhibitor (PPI) as the sole active ingredient, and
(ii) a core material containing the PPI and in the form of pellets, wherein:
(A) a first population of the pellets gives a delayed release pulse of the PPI, wherein pellets of this first population have the following sequence of layers on the core material:
A1—a delay release modifying layer comprising a water soluble polymer(s), talc and a hydrophobizing agent;
A2—a lag time controlling layer comprising a high viscosity water soluble polymer;
A3—an optional subcoating layer; and
A4—an outer enteric coating layer; and
(B) a second population of the pellets gives an immediate release pulse of the PPI, wherein pellets of this second population have the following sequence of layer(s) on the core material:
B1—an optional subcoating layer; and
B2—an outer enteric coating layer.

2. An oral solid pharmaceutical dosage form comprising
(i) an acid sensitive proton pump inhibitor (PPI) as the sole active ingredient, and
(ii) a core material containing the PPI and in the form of pellets, wherein each pellet gives a delayed release pulse of the PPI and an immediate release pulse of the PPI and has the following sequence of layers on the core material:
(1) a delay release modifying layer comprising a water soluble polymer(s), talc and a hydrophobizing agent;
(2) a lag time controlling layer comprising a high viscosity water soluble polymer;
(3) a layer comprising the second portion of the PPI;
(4) an optional subcoating layer; and
(5) an outer enteric coating layer.

3. The oral pharmaceutical dosage form according to claim 1 or 2, wherein the dosage form is a capsule.

4. The oral pharmaceutical dosage form according to claim 1 or 2, wherein the dosage form is a sachet.

5. The oral pharmaceutical dosage form according to claim 1, wherein the pellets giving an immediate release pulse of the PPI are in the form of one or more tablet(s).

6. The oral pharmaceutical dosage form according to claim 1 or 2, wherein the acid sensitive proton pump inhibitor is an alkaline salt of esomeprazole.

7. The oral pharmaceutical dosage form according to claim 1 or 2, wherein the acid sensitive proton pump inhibitor is esomeprazole magnesium.

8. The oral pharmaceutical dosage form according to claim 1 or 2, wherein the acid sensitive proton pump inhibitor is omeprazole magnesium.

9. The oral pharmaceutical dosage form according to claim 1 or 2, wherein the delayed release pulse has a lag time in the range of 1-10 hours.

10. The oral pharmaceutical dosage form according to claim 9, wherein the lag time is in the range of 2-8 hours.

11. The oral pharmaceutical dosage form according to claim 1 or 2, wherein the lag time controlling layer consists essentially of the high viscosity water soluble polymer.

12. The oral pharmaceutical dosage form according to claim 1 or 2 wherein the water soluble polymer of the lag time controlling layer is a high viscosity hydroxypropyl methyl cellulose or a high viscosity hydroxyethyl cellulose.

13. The oral pharmaceutical dosage form according to claim 12, wherein the high viscosity hydroxypropyl methyl cellulose or hydroxyethyl cellulose gives a pH of between 7.0-9.0 when measured according to Pharmacopoeia Europa.

14. The oral pharmaceutical dosage form according to claim 1 or 2 wherein the delay release modifying layer comprises one or more water soluble polymers, talc and a hydrophobizing agent selected from the group consisting of Mg-stearate, glyceryl behenate, and sodium stearyl fumarate.

15. The oral pharmaceutical dosage form according to claim 1 or 2 wherein the delay release modifying layer consists essentially of hydroxypropyl cellulose having a hydroxypropyl content in the range of 50-90% and a viscosity below 180 cps, talc and Mg-Stearate.

16. A process for preparing an oral pharmaceutical dosage form according to claim 1, wherein the dosage form comprises (i) an acid sensitive proton pump inhibitor (PPI) as the sole active ingredient, and (ii) a core material containing the PPI and in the form of pellets, the process comprising the following steps:
(a) preparing a first population of pellets containing the PPI;
(b) coating the pellets obtained in step a with a delay release modifying layer;
(c) coating the pellets obtained in step (b) with a lag time controlling layer comprising a high viscosity water soluble polymer;
(d) optionally applying a subcoating layer to the pellets obtained in step (c);
(e) coating the pellets obtained in step (c) or (d) with an outer enteric coating to obtain the first population of pellets giving a delayed release pulse of the PPI;
(f) mixing the first population of pellets obtained in step (e) with a second population of pellets containing the PPI and having an outer enteric coating and an optional subcoating layer, wherein the second population of pellets gives an immediate release of the PPI, and
(g) formulating the mixture of pellets obtained from step (f) into the dosage form.

17. A process for preparing an oral pharmaceutical dosage form according to claim 2, wherein the dosage form comprises (i) an acid sensitive proton pump inhibitor (PPI) as the sole active and (ii) a core material containing the PPI and in the form of pellets, the process comprising the following steps:
(a) preparing the pellets containing a first portion of the PPI;
(b) coating the pellets obtained in step (a) with a delay release modifying layer;
(c) coating the pellets obtained in step (b) with a lag time controlling layer comprising a high viscosity water soluble polymer;
(d) coating the pellets obtained in step (c) with a layer comprising a second portion of the PPI;
(e) optionally coating the pellets obtained in step d with a subcoating layer;
(f) coating the pellets obtained in step (d) or (e) with an outer enteric coating; and
(g) formulating the enteric coated pellets obtained in step (f) into the dosage form.

18. The process according to claim 16, wherein the second population of pellets is in the form of one or more tablet(s).

19. The process according to claim 16 or 17, wherein the step of coating the pellets with the lag time controlling layer is performed by applying a dispersion of the high viscosity water soluble polymer prepared by the steps of:
a) dispersing the high viscosity water soluble polymer in a non-solvent; and
b) adding an aqueous liquid or water to form a hydrated form of the dispersed polymer particles.

20. The process according to claim 16 or 17, wherein the delay release modifying layer consists essentially of hydroxypropyl cellulose, talc and Mg-Stearate.

21. The process according to claim 16 or 17, wherein the delayed release pulse has a lag time in the range of 1-10 hours.

22. A method for improving inhibition of gastric acid secretion, the method comprising administering an oral pharmaceutical dosage form as defined in any one of claims 1 or 2 to a patient in need thereof.

23. (canceled)

24. The process according to claim 16 or 17, wherein the dosage form is a capsule, sachet, or multiple unit pellets system tablet.
